(19) United States
(12) Patent Application Publication
(10) Pub. No.: US 2019/0275022 A1
(43) Pub. Date: Sep. 12, 2019

(54) METHODS, COMPOSITIONS, AND COMPOUNDS FOR TREATMENT OF DERMATOLOGICAL AND OCULAR CONDITIONS

(71) Applicant: Allergan, Inc., Irvine, CA (US)

(72) Inventors: Mohammed DIBAS, Corona, CA (US); John DONELLO, Dana Point, CA (US)

(21) Appl. No.: 16/319,249

(22) PCT Filed: Jul. 19, 2017
(86) PCT No.: PCT/US2017/042854

§ 371 Date: Jan. 18, 2019

Related U.S. Application Data
(60) Provisional application No. 62/364,547, filed on Jul. 20, 2016.

(51) Int. Cl. A61K 31/4412; A61P 27/02

(52) U.S. Cl. CPC A61K 31/4412; A61K 9/0048; A61P 27/02

(57) ABSTRACT

Described herein are methods, compositions, and compounds for the treatment of dermatological and ocular conditions.

### CROSS-REFERENCE TO RELATED APPLICATIONS

This application claims priority to, and the benefit of, U.S. Provisional Patent Application 62/364,547 filed on Jul. 20, 2016, which is hereby incorporated by reference in its entirety.

### FIELD

The present disclosure relates generally to the field of pharmaceutical treatment of dermatological and ocular conditions. The disclosure relates in particular to methods, compositions, and compounds for the treatment of dermatological and ocular conditions.

### BACKGROUND

Dermatological and inflammatory conditions can have a serious impact on the quality of life of an individual suffering from said conditions.

For example, psoriasis is an inflammatory disease characterized most commonly as well-circumscribed, erythematous papules and plaques covered with silvery scales that can form on the skin. The most common subtype of psoriasis is plaque psoriasis, however additional subtypes of psoriasis include guttate psoriasis, erythodermic psoriasis, general pustular psoriasis, pustular psoriasis of the palms and soles, inverse psoriasis, acrodermatitis continua of Hallopeau, and palmoplantar psoriasis. Psoriasis is rarely life-threatening, but it can affect a patient's self-image. Besides the patient's appearance, the sheer amount of time required to treat extensive skin or scalp lesions and to maintain clothing and bedding may adversely affect quality of life.

Similarly, dry eye disease (keratoconjunctivitis sicca, or KCS) is a chronic, bilateral desiccation of the conjunctiva and cornea due to an inadequate tear film with symptoms that can include itching, burning, irritation, and photophobia. Broadly, there are two types of dry eye disease based on their etiology: 1) aqueous tear-deficient keratoconjunctivitis sicca caused by inadequate tear volume, and 2) Evaporative keratoconjunctivitis sicca (more common) is caused by accelerated tear evaporation due to poor tear quality. Inflammation has been shown to be the key in the pathogenesis of this syndrome, as it seems to be the cause and the consequence of dry eye (Kyiomionis et al. Treatment of chronic dry eye: focus on cyclosporine. Clinical Ophthalmology 2000, 2, 829-836). Currently, the drug cyclosporine has been approved for the treatment of dry eye in patients whose tear production is presumed to be suppressed due to ocular inflammation.

Accordingly, there is a need for methods, compositions, and compounds for the treatment of dermatological and ocular conditions.

### SUMMARY

The present disclosure provides methods, compositions, and compounds for the treatment of dermatological and ocular conditions.

In a one aspect, described herein is a method of treating a dermatological condition or ocular condition in an individual in need thereof, the method comprising administering an effective amount of a compound of Formula Ia:

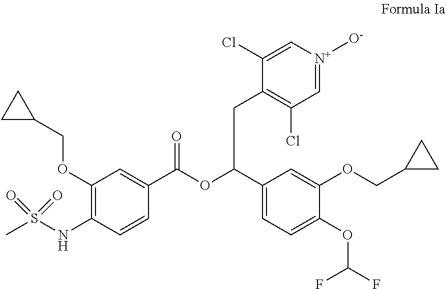 [SMILES: CS(=O)(=O)Nc1ccc(C(=O)OC(Cc2c(Cl)c[n+]([O-])cc2Cl)c2ccc(OC(F)F)c(OCC3CC3)c2)cc1OCC1CC1]

to the individual in need thereof.

Applicants have shown that the compound of Formula Ia, and in particular the compound of Formula I:

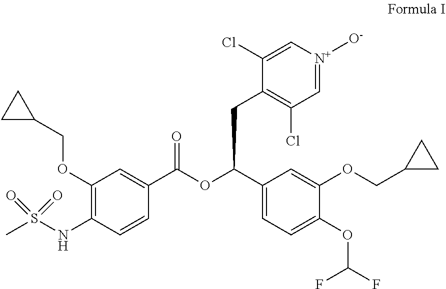 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

(also referred to herein as Compound A and also known as CHF-6001; see U.S. Pat. No. 8,440,834) surprisingly demonstrates better and more rapid onset of efficacy in dermal and ocular models of inflammation when compared to other PDE4 inhibitors, and also shows lower systemic exposure when compared to other PDE4 inhibitors. Given its low systemic exposure, this compound represents a safer PDE4 inhibitor. Targeting PDE4 with the highly selective compound of Formula I would also be expected to have benefits of reduced side effects as compared to approved PDE4 inhibitors. Therefore, the compound of Formula I would be expected to have a wide variety of therapeutic benefit in inflammatory disorders especially in the eye and skin.

Some example embodiments are listed below.

Example embodiment 1: a method of treating a dermatological condition or ocular condition in an individual in need thereof, the method comprising administering an effective amount of a compound of Formula Ia:

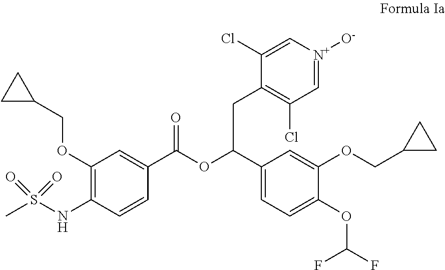 [SMILES: CS(=O)(=O)Nc1ccc(C(=O)OC(Cc2c(Cl)c[n+]([O-])cc2Cl)c2ccc(OC(F)F)c(OCC3CC3)c2)cc1OCC1CC1]

to the individual in need thereof.

Example embodiment 2: the method of example embodiment 1, wherein the dermatological condition is selected from the group consisting of atopic dermatitis, acne, psoriasis, seborrheic dermatitis, actinic keratosis, actinic keratosis, melasma, post-inflammatory hyperpigmentation, pruritus, and rosacea.

Example embodiment 3: the method of example embodiment 2, wherein the dermatological condition is psoriasis.

Example embodiment 4: the method of example embodiment 1, wherein the ocular condition is selected from the group consisting of uveitis, dry eye disease, keratitis, allergic eye disease, infectious keratitis, herpetic keratitis, retinitis, choroiditis, Behcet's disease, and wet and dry age-related macular degeneration (ARMD).

Example embodiment 5: the method of example embodiment 4, wherein the ocular condition is dry eye disease.

Example embodiment 6: the method of example embodiment 5, wherein the dry eye disease is dry eye disease in which tear production is suppressed due to ocular inflammation.

Example embodiment 7: the method of any one of example embodiments 1 to 6, wherein the administering of the compound of Formula Ia reduces inflammation associated with the dermatological condition or the ocular condition.

Example embodiment 8: the method of any one of example embodiments 1 to 7, wherein the compound of Formula Ia is administered topically.

Example embodiment 9: the method of any one of example embodiments 1 to 8, wherein the compound of Formula Ia has the structure:

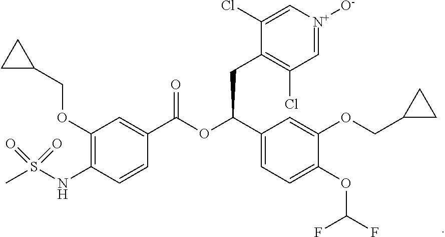 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

Example embodiment 10: use of a compound of Formula Ia:

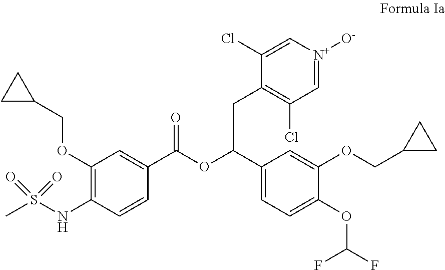 [SMILES: CS(=O)(=O)Nc1ccc(C(=O)OC(Cc2c(Cl)c[n+]([O-])cc2Cl)c2ccc(OC(F)F)c(OCC3CC3)c2)cc1OCC1CC1]

in the manufacture of a medicament for the treatment of a dermatological condition or ocular condition.

Example embodiment 11: the use according to example embodiment 10, wherein the dermatological condition is selected from the group consisting of atopic dermatitis, acne, psoriasis, seborrheic dermatitis, actinic keratosis, actinic keratosis, melasma, post-inflammatory hyperpigmentation, pruritus, and rosacea.

Example embodiment 12: the use according to example embodiment 11, wherein the dermatological condition is psoriasis.

Example embodiment 13: the use according to example embodiment 10, wherein the ocular condition is selected from the group consisting of uveitis, dry eye disease, keratitis, allergic eye disease, infectious keratitis, herpetic keratitis, retinitis, choroiditis, Behcet's disease, and wet and dry age-related macular degeneration (ARMD).

Example embodiment 14: the use according to example embodiment 13, wherein the ocular condition is dry eye disease.

Example embodiment 15: the method of example embodiment 14, wherein the dry eye disease is dry eye disease in which tear production is suppressed due to ocular inflammation.

Example embodiment 16: the use of any one of example embodiments 10 to 15, wherein the medicament, when administered to an individual in need of treatment of a dermatological condition or inflammatory condition, reduces inflammation associated with the dermatological condition or the ocular condition in the individual.

Example embodiment 17: the use according to any one of example embodiments 10 to 16, wherein the medicament, when administered to an individual in need of treatment of a dermatological condition or inflammatory condition, is administered topically.

Example embodiment 18: the use according to any one of example embodiments 10 to 17, wherein the compound of Formula Ia has the structure:

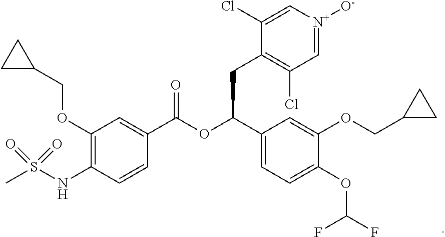 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

Example embodiment 19: use of the compound of Formula Ia:

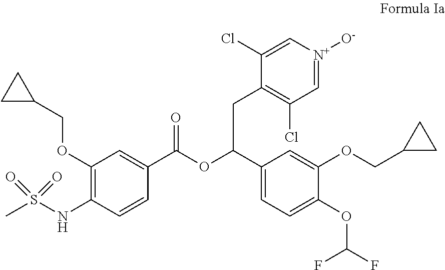 [SMILES: CS(=O)(=O)Nc1ccc(C(=O)OC(Cc2c(Cl)c[n+]([O-])cc2Cl)c2ccc(OC(F)F)c(OCC3CC3)c2)cc1OCC1CC1]

in the treatment of a dermatological condition or ocular condition in an individual in need thereof.

Example embodiment 20: the use according to example embodiment 19, wherein the dermatological condition is selected from the group consisting of atopic dermatitis, acne, psoriasis, seborrheic dermatitis, actinic keratosis, actinic keratosis, melasma, post-inflammatory hyperpigmentation, pruritus, and rosacea.

Example embodiment 21: the use according to example embodiment 20, wherein the dermatological condition is psoriasis.

Example embodiment 22: the use according to example embodiment 19, wherein the ocular condition is selected from the group consisting of uveitis, dry eye disease, keratitis, allergic eye disease, infectious keratitis, herpetic keratitis, retinitis, choroiditis, Behcet's disease, and wet and dry age-related macular degeneration (ARMD).

Example embodiment 23: the use according to example embodiment 22, wherein the ocular condition is dry eye disease.

Example embodiment 24: the method of example embodiment 23, wherein the dry eye disease is dry eye disease in which tear production is suppressed due to ocular inflammation.

Example embodiment 25: the use according to any one of example embodiments 19 to 24, wherein the use of the compound of Formula I in the treatment of the dermatological condition or ocular condition in an individual in need thereof reduces inflammation associated with the dermatological condition or the ocular condition.

Example embodiment 26: the use according to any one of example embodiments 19 to 25, wherein the compound of Formula I in the treatment of the dermatological condition or ocular condition in an individual in need thereof is administered topically.

Example embodiment 27: the use according to any one of example embodiments 19 to 26, wherein the compound of Formula Ia has the structure:

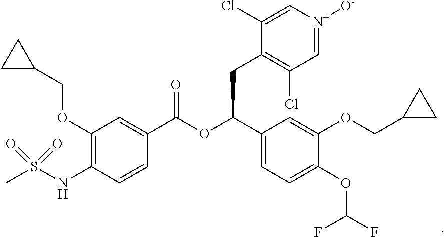 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

Example embodiment 28: the compound of Formula Ia:

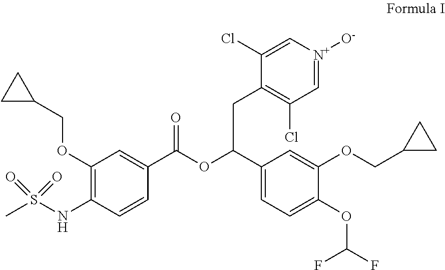 [SMILES: CS(=O)(=O)Nc1ccc(C(=O)OC(Cc2c(Cl)c[n+]([O-])cc2Cl)c2ccc(OC(F)F)c(OCC3CC3)c2)cc1OCC1CC1]

for use in the treatment of a dermatological condition or ocular condition in an individual in need thereof.

Example embodiment 29: the compound for use according to example embodiment 28, wherein the dermatological condition is selected from the group consisting of atopic dermatitis, acne, psoriasis, seborrheic dermatitis, actinic keratosis, actinic keratosis, melasma, post-inflammatory hyperpigmentation, pruritus, and rosacea.

Example embodiment 30: the compound for use according to example embodiment 29, wherein the dermatological condition is psoriasis.

Example embodiment 31: the compound for use according to example embodiment 28, wherein the ocular condition is selected from the group consisting of uveitis, dry eye disease, keratitis, allergic eye disease, infectious keratitis, herpetic keratitis, retinitis, choroiditis, Behcet's disease, and wet and dry age-related macular degeneration (ARMD).

Example embodiment 32: the compound for use according to example embodiment 31, wherein the ocular condition is dry eye disease.

Example embodiment 33: the compound for use according to example embodiment 32, wherein the dry eye disease is dry eye disease in which tear production is suppressed due to ocular inflammation.

Example embodiment 34: the compound for use according to any one of example embodiments 28 to 33, wherein in the treatment of a dermatological condition or ocular condition in an individual in need thereof, administration of the compound of Formula I reduces inflammation associated with the dermatological condition or the ocular condition.

Example embodiment 35: the compound for use according to any one of example embodiments 28 to 34, wherein in the treatment of a dermatological condition or ocular condition in an individual in need thereof, the compound of Formula I is administered topically.

Example embodiment 36: the compound for use according to any one of example embodiments 28 to 35, wherein the compound of Formula Ia has the structure:

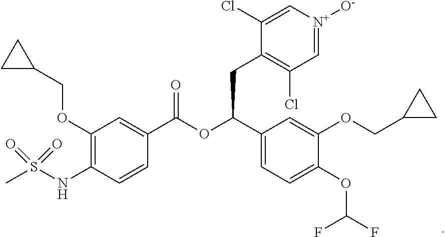 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

Example embodiment 37: a method of treating a dermatological condition or ocular condition in an individual in need thereof using a compound of Formula Ia:

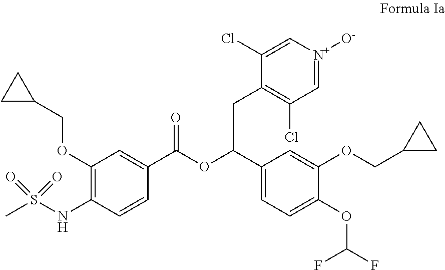 [SMILES: CS(=O)(=O)Nc1ccc(C(=O)OC(Cc2c(Cl)c[n+]([O-])cc2Cl)c2ccc(OC(F)F)c(OCC3CC3)c2)cc1OCC1CC1]

substantially as described herein.

Example embodiment 38: a method of treating a dermatological condition or ocular condition substantially as described herein.

Example embodiment 39: use of the compound of Formula Ia:

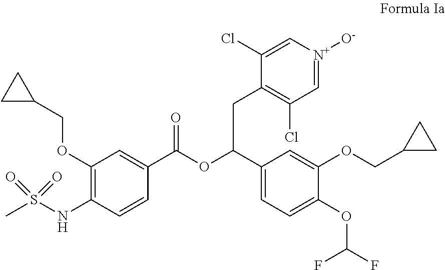 [SMILES: CS(=O)(=O)Nc1ccc(C(=O)OC(Cc2c(Cl)c[n+]([O-])cc2Cl)c2ccc(OC(F)F)c(OCC3CC3)c2)cc1OCC1CC1]

substantially as described herein.

Example embodiment 40: the compound of Formula Ia:

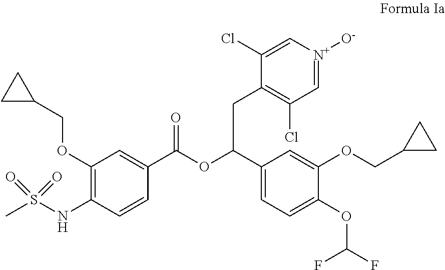 [SMILES: CS(=O)(=O)Nc1ccc(C(=O)OC(Cc2c(Cl)c[n+]([O-])cc2Cl)c2ccc(OC(F)F)c(OCC3CC3)c2)cc1OCC1CC1]

for uses as substantially described herein.

### DESCRIPTION OF THE DRAWINGS

FIG. 1 shows a graph of the results of topical administration of the compound of Formula I (Compound A) in an animal model of dermatitis as compared to other PDE4 inhibitors. The vertical axis shows the scoring atopic dermatitis (SCORAD) scale used to assess the extent and severity of eczema/atopic dermatitis.

FIG. 2 shows a graphical comparison of efficacy of the compound of Formula I (Compound A) as compared to the efficacy of the PDE4 inhibitors apremilast and roflumilast.

FIG. 3 shows a graphical comparison of the efficacy and systemic exposure of the topical administration of the compound of Formula I (Compound A) at various doses as compared to the PDE4 inhibitor AN-2728.

FIG. 4 shows a graph of the percentage of cells (left graph) and protein (right graph) in the aqueous humor when the activity compound of Formula I (Compound A) is compared to the PDE4 inhibitor AN-2728 in the lipopolysaccharide (LPS)-induced ocular inflammation model.

### DETAILED DESCRIPTION

It is to be understood that both the foregoing general description and the following detailed description are exemplary and explanatory only and are not restrictive of the invention disclosed and claimed herein. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

Unless specific definitions are provided, the nomenclatures utilized in connection with the laboratory procedures and techniques of analytical chemistry, synthetic organic and inorganic chemistry described herein are those known in the art. Standard chemical symbols are used interchangeably with the full names represented by such symbols. Thus, for example, the terms “hydrogen” and “H” are understood to have identical meaning. Standard techniques can be used for chemical syntheses, chemical analyses, and formulation.

Disclosed herein are methods for the treatment of dermatological or ocular conditions in an individual in need thereof.

In some embodiments, the method can be a method of treating a dermatological condition or ocular condition in an individual in need thereof, the method comprising administering an effective amount of a compound of Formula Ia:

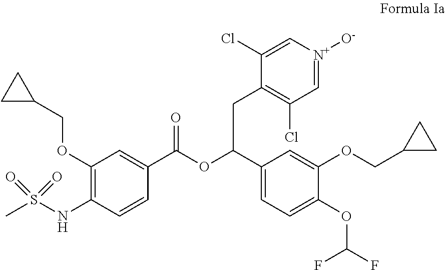 [SMILES: CS(=O)(=O)Nc1ccc(C(=O)OC(Cc2c(Cl)c[n+]([O-])cc2Cl)c2ccc(OC(F)F)c(OCC3CC3)c2)cc1OCC1CC1]

to the individual in need thereof.

In some embodiments, the method can be a method of treating a dermatological condition or ocular condition in an individual in need thereof, the method comprising administering an effective amount of a compound of Formula I:

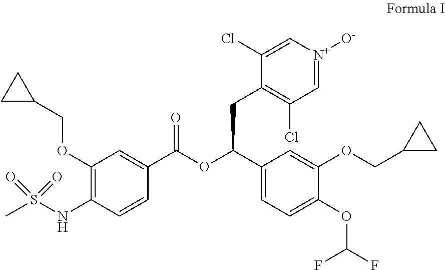 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

to the individual in need thereof.

The terms “individual” and “subject” as used herein (e.g. as in “individual in need thereof” or “subject in need thereof”) refer to a human or non-human animal.

The term “dermatological condition” as used herein refers to a disease or condition affecting the skin, including those diseases or conditions affecting the skin which involve inflammation as part of their etiology and/or pathophysiology (e.g. inflammatory dermatological conditions such as ones in which the phosphodiesterase 4 (PDE4) enzyme is involved or believed to be involved in their etiology and/or pathophysiology). Some dermatological conditions treatable by the methods, compositions, and compounds described herein include, for example, atopic dermatitis, acne, psoriasis, seborrheic dermatitis, actinic keratosis, actinic keratosis, melasma, post-inflammatory hyperpigmentation, rosacea, pruritus, and others identifiable to a skilled person upon a reading of the present disclosure.

In some embodiments, the dermatological condition is psoriasis. In some embodiments, the dermatological condition is a subtype of psoriasis such as, for example, plaque psoriasis, guttate psoriasis, erythodermic psoriasis, general pustular psoriasis, pustular psoriasis of the palms and soles, inverse psoriasis, acrodermatitis continua of Hallopeau, and palmoplantar psoriasis.

The term “ocular condition” as used herein refers to a disease or condition affecting the eye, including those diseases or conditions affecting the eye which involve inflammation as part of their etiology and/or pathophysiology (e.g. inflammatory ocular conditions such as ones in which the phosphodiesterase 4 (PDE4) enzyme is involved or believed to be involved in their etiology and/or pathophysiology). Some ocular conditions treatable by the methods, compositions, and compounds described herein include, for example, uveitis, dry eye disease, keratitis, allergic eye disease, infectious keratitis, herpetic keratitis, retinitis, choroiditis, Behcet's disease, wet and dry age-related macular degeneration (ARMD), and others identifiable to a skilled person upon a reading of the present disclosure.

In some embodiments, the ocular condition is dry eye disease. In some embodiments, the ocular condition is dry eye disease in which tear production is, or is presumed to be, suppressed due to ocular inflammation.

Without wishing to be bound by theory, Applicants believe that methods, compositions, and compounds described herein operate by inhibiting the phosphodiesterase 4 (PDE4) enzyme, which is highly expressed in inflammatory and immune cells regulating intracellular second messenger cAMP levels and plays a critical role in ocular and dermatological inflammatory diseases (Bjørgo E et al. Phosphodiesterases as targets for modulating T-cell responses. Handb. Exp. Pharmacol. 2011, 204, 345-363). Accordingly, the methods, compositions, and compounds described herein are expected to be useful in the treatment (e.g. reduction) of inflammation associated with dermatological diseases, disorders, conditions, and/or events such as, for example, atopic dermatitis, acne, psoriasis (including, for example, plaque psoriasis, guttate psoriasis, erythodermic psoriasis, general pustular psoriasis, pustular psoriasis of the palms and soles, inverse psoriasis, acrodermatitis continua of Hallopeau, and palmoplantar psoriasis), seborrheic dermatitis, pruritus, basal cell carcinoma, squamous cell carcinoma, melanoma, viral warts, photoaging, photodamage, melasma, post-inflammatory hyperpigmentation, dermal wounds, dermal wound healing, hypertrophic scars, keloids, burns, rosacea, disorders of pigmentation (e.g. acanthosis nigricans, age spots, albinism, hyperpigmentation, incontinentia pigmenti, lentigines, McCune-Albright syndrome, melasma, pityriasis alba, progressive pigmentary purpura, Dowling-Degos disease, Griscelli syndrome, Hermansky-Pudlak syndrome, histiocytosis-lymphadenopathy plus syndrome, incontinentia pigmenti, multiple lentigines syndrome, Naegeli-Franceschetti-Jadassohn syndrome/dermatopathia pigmentosa reticularis, oculocutaneous albinism, piebaldism, Tietz syndrome, Waardenburg syndrome, xeroderma pigmentosum, and others identifiable to a skilled person upon a reading of the present disclosure), alopecia (scarring and non-scarring forms), and other dermatological diseases, disorders, conditions, and/or events identifiable to a skilled person upon a reading of the present disclosure.

Similarly, the methods, compositions, and compounds described herein are expected to be useful in the treatment (e.g. reduction) of inflammation associated with ocular diseases, disorders, conditions, and/or events such as, for example, uveitis, dry eye disease (including, for example, dry eye disease in which tear production is, or is presumed to be, suppressed due to ocular inflammation), keratitis, allergic eye disease, infectious keratitis, herpetic keratitis, corneal angiogenesis, lymphangiogenesis, retinitis, choroiditis (e.g. acute multifocal placoid pigment epitheliopathy, multifocal choroiditis, panuveitis, and others identifiable to a skilled person upon a reading of the present disclosure), Behcet's disease, post-surgical inflammation, corneal wound healing, conditions caused by laser (e.g. LASIK, corneal inlays, presbyopia correction, and others identifiable to a skilled person upon a reading of the present disclosure), conditions caused by photodynamic therapy, wet and dry age-related macular degeneration, and other ocular diseases, disorders, conditions, and/or events identifiable to a skilled person upon a reading of the present disclosure.

The term “administering” as used herein refers to introduction of a substance (e.g. the compound of Formulas described herein) into a body of a subject and/or application of a substance onto the body of a subject by a particular route. Routes of administration would be identifiable to a skilled person and include, for example, oral administration, parenteral administration (e.g. subcutaneous injection, intramuscular injection, and intravenous injection and other parenteral administration routes identifiable to a skilled person upon a reading of the present disclosure), sublingual administration, buccal administration, rectal administration, ocular administration (e.g. topical administration, suprachoroidal administration, periocular administration, intracameral injection, intravitreal injection, sub-Tenon injection, sub-conjuntivital, ocular implants such as drug delivery system (DDS) implants, and other ocular administration routes identifiable to a skilled person upon a reading of the present disclosure), otic administration, inhalation routes (e.g. inhaling a mist containing the substance though the mouth or nose and other inhalation routes identifiable to a skilled person upon a reading of the present disclosure), topical administration, transdermal administration (e.g. via transdermal patches and other transdermal routes identifiable to a skilled person upon a reading of the present disclosure), administration via an implant device, and others identifiable to a skilled person upon a reading of the present disclosure.

Administration can be “local” when the compound is administered to a particular localized region of the body and only that region near the site of administration is exposed to the compound (e.g. topical application or subcutaneous application to a particular region of the subject's body).

Similarly, administration can be “systemic” when the compound is administered such that the compound is exposed throughout the subject's body and may be found in one or more regions distant from the site of administration (e.g. orally or intravenously administering the compound such that the compound will be distributed in the blood and throughout various tissues and/or body regions resulting treatment of the dermatological and/or ocular disorder at those tissues and/or regions).

In some embodiments, the compounds (e.g. the compound of Formula I) can be administered by administering pharmaceutical compositions to a subject. The pharmaceutical compositions can contain at least one compound described herein in a pharmaceutically acceptable carrier thereof and can in some embodiments contain one or more pharmaceutically acceptable excipients. The phrase “pharmaceutically acceptable” means the carrier, diluent or excipient is compatible with the other ingredients of the formulation and not deleterious to the recipient thereof.

In particular, the pharmaceutical compositions of the compounds described herein that can be used in the methods described herein can be ophthalmically acceptable compositions. The term “ophthalmically acceptable” as used herein refers to a composition formulated such that it can be administered to or into the eye—without undue toxicity, incompatibility, instability, allergic response, irritation, and the like—by routes amenable to ocular administration (e.g. topical administration, suprachoroidal administration, periocular administration, intracameral injection, intravitreal injection, sub-conjuntivital, sub-Tenon injection, ocular implants such as drug delivery system (DDS) implants, and other ocular administration routes identifiable to a skilled person upon a reading of the present disclosure). The comfort should be maximized as much as possible, although sometimes formulation considerations (e.g. stability) may necessitate less than optimal comfort. In the case that comfort cannot be maximized, the liquid should be formulated such that the liquid is tolerable to the patient for administration to the eye. Additionally, an ophthalmically acceptable composition can be packaged for single use, or can contain a preservative to prevent contamination over multiple uses.

Similarly, the pharmaceutical compositions of the compounds described herein that can be used in the methods described herein can be dermatologically acceptable compositions. The term “dermatologically acceptable” as used herein refers to a composition formulated such that it can be administered to or into the skin—without undue toxicity, incompatibility, instability, allergic response, irritation, and the like—by routes amenable to dermal administration (e.g. topical administration, injection (e.g. subcutaneous injection) dermal administration devices such as patches, dermal and transdermal drug delivery systems (DDS), iontophoresis devices, ultrasound devices, microneedle devices and other devices identifiable to a skilled person upon a reading of the present disclosure, and other dermal delivery routes identifiable to a skilled person upon a reading of the present disclosure). The comfort should be maximized as much as possible, although sometimes formulation considerations (e.g. stability) may necessitate less than optimal comfort. In the case that comfort cannot be maximized, the liquid should be formulated such that the liquid is tolerable to the patient for dermatological use. Additionally, a dermatologically acceptable composition can be packaged for single use, or can contain a preservative to prevent contamination over multiple uses.

Pharmaceutical compositions of the compounds described herein can be used in the form of a solid, a solution, a suspension, an emulsion, a dispersion, a patch, a micelle, a liposome, and others identifiable to a skilled person upon a reading of the present disclosure, wherein the resulting composition contains one or more compounds described herein, as an active ingredient, in admixture with an organic or inorganic carrier or excipient suitable for enteral or parenteral applications. Compounds described herein can be combined, for example, with non-toxic, pharmaceutically acceptable carriers known to a skilled person for tablets, pellets, capsules, suppositories, solutions, emulsions, suspensions, and any other form suitable for use identifiable to a skilled person. Some carriers which can be used can include glucose, lactose, gum acacia, gelatin, mannitol, starch paste, magnesium trisilicate, talc, corn starch, keratin, colloidal silica, potato starch, urea, medium chain length triglycerides, dextrans, and other carriers identifiable to a skilled person suitable for use in manufacturing preparations, in solid, semisolid, or liquid form. In addition, auxiliary agents, stabilizing agents, thickening agents, coloring agents, and perfumes can be used. Compounds described herein can be included in the pharmaceutical composition in an amount sufficient to produce the desired effect upon the subject's condition (e.g. treatment of the dermatological condition or ocular condition).

Pharmaceutical compositions containing compounds described herein can be in a form suitable for oral use, for example, as tablets, troches, lozenges, aqueous or oily suspensions, dispersible powders or granules, emulsions, hard or soft capsules, or syrups or elixirs, as well as others identifiable to a skilled person upon a reading of the present disclosure. Compositions intended for oral use can be prepared according to any method known in the art, and others identifiable to a skilled person upon a reading of the present disclosure, for the manufacture of pharmaceutical compositions and such compositions can contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing compounds described herein in admixture with non-toxic pharmaceutically acceptable excipients can also be manufactured by known methods. The excipients used can be, for example, (1) inert diluents such as calcium carbonate, lactose, calcium phosphate or sodium phosphate, or others identifiable to a skilled person upon a reading of the present disclosure; (2) granulating and disintegrating agents such as corn starch, potato starch or alginic acid, or others identifiable to a skilled person upon a reading of the present disclosure; (3) binding agents such as gum tragacanth, corn starch, gelatin or acacia, or others identifiable to a skilled person upon a reading of the present disclosure, and (4) lubricating agents such as magnesium stearate, stearic acid or talc, or others identifiable to a skilled person upon a reading of the present disclosure. The tablets can be uncoated or they can be coated by known techniques identifiable to a skilled person to delay disintegration and absorption in the gastrointestinal tract and thereby provide a sustained action over a longer period. For example, a time delay material such as glyceryl monostearate or glyceryl distearate can be employed.

In some cases, formulations for oral use can be in the form of hard gelatin capsules wherein the compounds described herein are mixed with an inert solid diluent, for example, calcium carbonate, calcium phosphate or kaolin, or others identifiable to a skilled person upon a reading of the present disclosure. Formulations can also be in the form of soft gelatin capsules wherein the compounds described herein are mixed with water or an oil medium, for example, peanut oil, liquid paraffin or olive oil, or others identifiable to a skilled person upon a reading of the present disclosure.

The pharmaceutical compositions can be in the form of a sterile injectable suspension. This suspension can be formulated according to known methods identifiable to a skilled person using suitable dispersing or wetting agents and suspending agents. The sterile injectable preparation can also be a sterile injectable solution or suspension in a non-toxic parenterally acceptable diluent or solvent, for example, as a solution in 1,3-butanediol, or other non-toxic parenterally acceptable diluents or solvents identifiable to a skilled person. Sterile, fixed oils are conventionally employed as a solvent or suspending medium. For this purpose, a bland fixed oil can be employed including synthetic mono- or diglycerides, fatty acids (including oleic acid), naturally occurring vegetable oils like sesame oil, coconut oil, peanut oil, cottonseed oil, and others identifiable to a skilled person, or synthetic fatty vehicles like ethyl oleate and others identifiable to a skilled person. Buffers, preservatives, antioxidants, and others identifiable to a skilled person can be incorporated as required.

Pharmaceutical compositions containing compounds described herein can be in a form suitable for topical use, for example, as oily suspensions, as solutions or suspensions in aqueous liquids or non-aqueous liquids, or as oil-in-water or water-in-oil liquid emulsions. Pharmaceutical compositions can be prepared by combining a therapeutically effective amount of at least one compound described herein as an active ingredient with conventional topically acceptable pharmaceutical excipients and by preparation of unit dosage suitable for topical use.

The compounds described herein can also be administered in the form of suppositories for rectal administration of the compounds. These compositions can be prepared by mixing the compounds described herein with a suitable non-irritating excipient, such as, for example, cocoa butter, synthetic glyceride esters of polyethylene glycols, which are solid at ordinary temperatures, but liquefy and/or dissolve in the rectal cavity to release the compounds.

For ophthalmic application, ophthalmically acceptable compositions can be prepared using a physiological saline solution as a major vehicle. Ophthalmically acceptable compositions can be maintained at a comfortable pH with an appropriate buffer system. The compositions can also contain conventional, pharmaceutically acceptable excipients such as preservatives, stabilizers, surfactants, and others identifiable to a skilled person upon a reading of the present disclosure.

Preservatives that can be used in the ophthalmically acceptable compositions described herein can be, for example, benzalkonium chloride, stabilized oxychloro complex (e.g. PURITE®), chlorobutanol, thimerosal, phenylmercuric acetate, phenylmercuric nitrate, and other preservatives identifiable to a skilled person upon a reading of the present disclosure. A useful surfactant can be, for example, Tween 80, though other surfactants are identifiable a to a skilled person upon a reading of the present disclosure. Likewise, various useful vehicles can be used in the ophthalmically acceptable compositions described herein. These vehicles can be, for example, polyvinyl alcohol, povidone, hydroxypropyl methyl cellulose, poloxamers, carboxymethyl cellulose, hydroxyethyl cellulose, purified water, and others identifiable to a skilled person upon a reading of the present disclosure.

Tonicity adjustors can be added as needed or convenient. They can be, for example, salts (e.g. sodium chloride and potassium chloride), mannitol and glycerin, or any other suitable ophthalmically acceptable tonicity adjustor identifiable to a skilled person upon a reading of the present disclosure.

Various buffers and means for adjusting pH can be used so long as the resulting preparation is ophthalmically acceptable. Accordingly, buffers can include, for example, acetate buffers, citrate buffers, phosphate buffers, borate buffers, and other buffers identifiable to a skilled person upon a reading of the present disclosure. Acids or bases can be used to adjust the pH of these formulations as needed.

For dermatological application, the dermatologically acceptable compositions described herein can include one or more penetration enhancers. The term “penetration enhancer” as used herein refers to any agent that facilitates the transfer of active components to their site of action or maintains them at their site of action. Non-limiting examples of classes of appropriate penetration enhancers include alcohols, glycols, fatty acids, ethers, esters, occlusive agents and surface active agents. Representative examples of these classes are provided below.

Alcohols useful as penetration enhancers in the dermatologically acceptable compositions can be, for example, ethanol, propanol, N-propanol, isopropanol, butyl alcohol, octanol, benzyl alcohol, acetyl alcohol, and others identifiable to a skilled person upon a reading of the present disclosure. Fatty alcohols can be, for example, stearyl alcohol, oleyl alcohol, and others identifiable to a skilled person upon a reading of the present disclosure.

Glycols useful as penetration enhancers in the dermatologically acceptable compositions can be, for example, propyleneglycol, polyethyleneglycol, other low molecular weight glycols such as glycerol and thioglycerol, and other glycols identifiable to a skilled person upon a reading of the present disclosure.

Fatty acids, esters and ethers useful as penetration enhancers in the dermatologically acceptable compositions can be, for example, oleic acid, palmitoleic acid, straight chain C₄-C₂₀ saturated monocarboxylic and dicarboxylic acids, octanoic and decanoic acids, methyl laurate, ethyl oleate, polyethylene glycol monolaurate, propylene glycol monolaurate, propylene glycerol dilaurate, glycerol monolaurate, glycerol monooleate, isopropyl n-decanoate, octyldodecyl myristate, diethylene glycol monoethyl ether, diethylene glycol monomethyl ether and compounds wherein a C₂-C₄ alkane diol or triol is substituted with one or two fatty ether substituents, and other fatty acids, esters, and ethers identifiable to a skilled person upon a reading of the present disclosure.

Occlusive agents useful as penetration enhancers in the dermatologically acceptable compositions can be, for example, silicones, mineral oils and greases, long chain acids, animal fats and greases, vegetable fats and greases, water insoluble polymers, paraffin, paraffin oil, liquid paraffin, petrolatum, liquid petrolatum, white petrolatum, yellow petrolatum, microcrystalline wax, ceresin, and other occlusive agents identifiable to a skilled person upon a reading of the present disclosure.

Surface active agents useful as penetration enhancers in the dermatologically acceptable compositions can be, for example, polysorbate 20, 40, 60 and 80, TWEEN® (20, 40, 60, 80), POLOXAMER® (231, 182, 184), sodium dodecyl sulfate (SDS), lecithin, lysolecithin, nonylphenoxypolyoxyethylene, lysophosphatidylcholine, polyethylenglycol 400, polyoxyethylene ethers, polyglycol ether surfactants, DMSO, sodium laurate, sodium lauryl sulfate, cetyltrimethylammonium bromide, benzalkonium chloride, and other surface active agents identifiable to a skilled person upon a reading of the present disclosure.

The dermatologically acceptable compositions described herein can also include viscosity increasing agents. Appropriate viscosity increasing agents useful in the dermatologically acceptable compositions can be, for example, methylcellulose, polyvinyl alcohol, polyvinyl pyrrolidone, hyaluronic acid, chondroitin sulfate, and others identifiable to a skilled person upon a reading of the present disclosure.

The dermatologically acceptable compositions described herein can include preservatives such as, for example, benzyl alcohol, benzalkonium chloride, chlorhexidine, chlorobutanol, methyl-, propyl-, or butyl-parahydroxybenzoic acids, phenylmercuric salts (e.g., nitrate, chloride, acetate, borate, and others identifiable to a skilled person upon a reading of the present disclosure), betain, and other preservatives identifiable to a skilled person upon a reading of the present disclosure.

Various other additives can be included in the dermatologically acceptable compositions described herein in addition to those identified above. These can include, for example, antioxidants, astringents, perfumes, emollients, pigments, dyes, humectants, propellants, and sunscreen agents, as well as other classes of additives whose presence may be cosmetically, medicinally or otherwise desirable.

Since individual subjects can present a wide variation in the treatment requirements for the treatment of the dermatological disorder or ocular disorder, and since each compound has its unique characteristics, the precise mode of administration and dosage employed for each subject is left to the discretion of the practitioner.

In some embodiments, when the compounds described herein (e.g. the compound of Formula I) are part of a composition, the compounds are the only active ingredients which result in treatment of the dermatological condition or ocular condition and/or treatment (e.g. reduction) of inflammation associated with dermatological and/or ocular diseases, disorders, conditions, as described herein. The term “active ingredient” as used herein refers to a component which is responsible for the biological effect of treatment of the dermatological condition or ocular condition and/or treatment (e.g. reduction) of inflammation associated with dermatological and/or ocular diseases, disorders, conditions, as described herein, whereas the other components of the composition (e.g. excipients, carriers, and diluents) are not responsible for the biological effect of treatment of the dermatological condition or ocular condition and/or treatment (e.g. reduction) of inflammation associated with dermatological and/or ocular diseases, disorders, conditions, as described herein, even if they have other functions in the composition which are necessary or desired as part of the formulation (such as lubrication, flavoring, pH control, emulsification, and other functions other than treatment of the dermatological condition or ocular condition and/or treatment (e.g. reduction) of inflammation associated with dermatological and/or ocular diseases, disorders, conditions, as described herein).

The terms “effective amount” and “therapeutically effective amount” as used herein refer to an amount of a compound (e.g. the compound of Formula I) which will exert a therapeutic effect when administered to an individual. For example, a given amount of a compound will be an “effective amount” when administration of that amount of the compound results in the effective treatment of the dermatological condition or ocular condition and/or treatment (e.g. reduction) of inflammation associated with dermatological and/or ocular diseases, disorders, conditions, as described herein as determined by evaluation techniques identifiable to a skilled person upon a reading of the present disclosure.

The actual amount of the compound to be administered in any given case will be determined by a physician and/or other skilled person taking into account the relevant circumstances and/or factors, such as the cause, extent, and/or severity of the dermatological condition or ocular condition and/or inflammation associated with dermatological and/or ocular diseases, disorders, conditions, as described herein, as well as the age and weight of the patient, the patient's general physical condition, the route of administration and other circumstances and/or factors identifiable to a skilled person upon a reading of the present disclosure.

The therapeutically effective amount of the compounds described herein can be present in a pharmaceutical composition (e.g. for topical administration) at, for example, between about 0.01 and about 5% (w/v). In some embodiments, the therapeutically effective amount in the composition can be from about 0.001 to about 1%, about 0.001 to about 2%, about 0.001 to about 3%, and about 0.001 to about 4% (w/v). In other embodiments, the therapeutically effective amount in the composition can be from about 0.001 to about 1%, about 1 to about 2%, about 2 to about 3%, about 3 to about 4%, about 4 to about 5% (w/v).

In other embodiments, the therapeutically effective amount in the composition can be from about 0.001% to about 0.002%, about 0.002% to about 0.003%, about 0.003% to about 0.004%, about 0.004% to about 0.005%, about 0.005% to about 0.006%, about 0.006% to about 0.007%, about 0.007% to about 0.008%, about 0.008% to about 0.009%, about 0.009% to about 0.01%, about 0.001% to about 0.005%, about 0.005% to about 0.009%, about 0.009% to about 0.013%, about 0.013% to about 0.017%, about 0.017% to about 0.021%, about 0.021% to about 0.025%, about 0.025% to about 0.029%, about 0.029% to about 0.033%, about 0.033% to about 0.037%, about 0.037% to about 0.041%, about 0.041% to about 0.045%, about 0.045% to about 0.049%, about 0.049% to about 0.053%, about 0.053% to about 0.057%, about 0.057% to about 0.061%, about 0.061% to about 0.065%, about 0.065% to about 0.069%, about 0.069% to about 0.073%, about 0.073% to about 0.077%, about 0.077% to about 0.081%, about 0.081% to about 0.085%, about 0.085% to about 0.089%, about 0.089% to about 0.093%, about 0.093% to about 0.097%, about 0.097% to about 0.1%, about 0.001% to about 0.021%, about 0.021% to about 0.041%, about 0.041% to about 0.061%, about 0.061% to about 0.081%, about 0.081% to about 0.101%, about 0.101% to about 0.121%, about 0.121% to about 0.141%, about 0.141% to about 0.161%, about 0.161% to about 0.181%, about 0.181% to about 0.201%, about 0.201% to about 0.221%, about 0.221% to about 0.241%, about 0.241% to about 0.261%, about 0.261% to about 0.281%, about 0.281% to about 0.301%, about 0.301% to about 0.321%, about 0.321% to about 0.341%, about 0.341% to about 0.361%, about 0.361% to about 0.381%, about 0.381% to about 0.401%, about 0.401% to about 0.421%, about 0.421% to about 0.441%, about 0.441% to about 0.461%, about 0.461% to about 0.481%, about 0.481% to about 0.501%, about 0.501% to about 0.521%, about 0.521% to about 0.541%, about 0.541% to about 0.561%, about 0.561% to about 0.581%, about 0.581% to about 0.601%, about 0.601% to about 0.621%, about 0.621% to about 0.641%, about 0.641% to about 0.661%, about 0.661% to about 0.681%, about 0.681% to about 0.701%, about 0.701% to about 0.721%, about 0.721% to about 0.741%, about 0.741% to about 0.761%, about 0.761% to about 0.781%, about 0.781% to about 0.801%, about 0.801% to about 0.821%, about 0.821% to about 0.841%, about 0.841% to about 0.861%, about 0.861% to about 0.881%, about 0.881% to about 0.901%, about 0.901% to about 0.921%, about 0.921% to about 0.941%, about 0.941% to about 0.961%, about 0.961% to about 0.981%, about 0.981% to about 1%, about 0.001% to about 0.051%, about 0.051% to about 0.101%, about 0.101% to about 0.151%, about 0.151% to about 0.201%, about 0.201% to about 0.251%, about 0.251% to about 0.301%, about 0.301% to about 0.351%, about 0.351% to about 0.401%, about 0.401% to about 0.451%, about 0.451% to about 0.501%, about 0.501% to about 0.551%, about 0.551% to about 0.601%, about 0.601% to about 0.651%, about 0.651% to about 0.701%, about 0.701% to about 0.751%, about 0.751% to about 0.801%, about 0.801% to about 0.851%, about 0.851% to about 0.901%, about 0.901% to about 0.951%, about 0.951% to about 1.001%, about 1.001% to about 1.051%, about 1.051% to about 1.101%, about 1.101% to about 1.151%, about 1.151% to about 1.201%, about 1.201% to about 1.251%, about 1.251% to about 1.301%, about 1.301% to about 1.351%, about 1.351% to about 1.401%, about 1.401% to about 1.451%, about 1.451% to about 1.501%, about 1.501% to about 1.551%, about 1.551% to about 1.601%, about 1.601% to about 1.651%, about 1.651% to about 1.701%, about 1.701% to about 1.751%, about 1.751% to about 1.801%, about 1.801% to about 1.851%, about 1.851% to about 1.901%, about 1.901% to about 1.951%, about 1.951% to about 2.001%, about 2.001% to about 2.051%, about 2.051% to about 2.101%, about 2.101% to about 2.151%, about 2.151% to about 2.201%, about 2.201% to about 2.251%, about 2.251% to about 2.301%, about 2.301% to about 2.351%, about 2.351% to about 2.401%, about 2.401% to about 2.451%, about 2.451% to about 2.501%, about 2.501% to about 2.551%, about 2.551% to about 2.601%, about 2.601% to about 2.651%, about 2.651% to about 2.701%, about 2.701% to about 2.751%, about 2.751% to about 2.801%, about 2.801% to about 2.851%, about 2.851% to about 2.901%, about 2.901% to about 2.951%, about 2.951% to about 3.001%, about 3.001% to about 3.051%, about 3.051% to about 3.101%, about 3.101% to about 3.151%, about 3.151% to about 3.201%, about 3.201% to about 3.251%, about 3.251% to about 3.301%, about 3.301% to about 3.351%, about 3.351% to about 3.401%, about 3.401% to about 3.451%, about 3.451% to about 3.501%, about 3.501% to about 3.551%, about 3.551% to about 3.601%, about 3.601% to about 3.651%, about 3.651% to about 3.701%, about 3.701% to about 3.751%, about 3.751% to about 3.800%, about 3.800% to about 3.850%, about 3.850% to about 3.900%, about 3.900% to about 3.950%, about 3.950% to about 4.000%, about 4.000% to about 4.050%, about 4.050% to about 4.100%, about 4.100% to about 4.150%, about 4.150% to about 4.200%, about 4.200% to about 4.250%, about 4.250% to about 4.300%, about 4.300% to about 4.350%, about 4.350% to about 4.400%, about 4.400% to about 4.450%, about 4.450% to about 4.500%, about 4.500% to about 4.550%, about 4.550% to about 4.600%, about 4.600% to about 4.650%, about 4.650% to about 4.700%, about 4.700% to about 4.750%, about 4.750% to about 4.800%, about 4.800% to about 4.850%, about 4.850% to about 4.900%, about 4.900% to about 4.950%, and about 4.950% to about 5% (w/v).

Unless indicated otherwise herein, the term “about” is intended to include values (e.g., weight percentages) proximate to the recited range and/or value that are equivalent (e.g. bioequivalent) in terms of the functionality of the individual ingredient (e.g. active ingredient or excipient), the composition, or the embodiment. Furthermore, as will be understood by a skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

The compounds described herein can be administered according to a dosing frequency that is identifiable to a skilled person during a time period that is also identifiable to a skilled person. The term “dosing frequency” as used herein, refers to the number of times the compounds described herein are administered to a subject. Some dosing frequencies include administering the effective amount at discrete times during a day such as, for example, once a day (QD), twice a day (BID), three times a day (TID), four times a day (QID), and others identifiable to a skilled person. Other dosing frequencies include continuous dosing, for example by intravenous infusion, use of a drug pump, use of a transdermal patch, or other methods of continuous dosing identifiable to a skilled person upon a reading of the present disclosure.

The compounds described herein can be administered at a desired dosing frequency for a time period identifiable to a skilled person. For example, a compound can be administered once or twice a day (or at another dosing frequency identifiable to a skilled person) for a set period of time (e.g. seven to fourteen days, two to four weeks, one to six months, or for another time period identifiable to a skilled person). As another example, a compound can be administered once or twice a day (or at another dosing frequency identifiable to a skilled person) for a non-predetermined period of time. A skilled person can determine at various points during the period of time if the administration of the compound is to be continued (e.g., if a desired outcome such as a particular extent of treatment of the dermatological condition or ocular condition and/or inflammation associated with dermatological and/or ocular diseases, disorders, conditions, as described herein has been achieved and administration of the compound is not required and/or desired anymore).

Some compounds described in here can exist as different stereoisomers (e.g. diastereomers, enantiomers, geometrical isomers, atropisomers, and others identifiable to a skilled person upon a reading of the present disclosure). In particular, some compounds described herein have at least one asymmetric center in their structure. This asymmetric center can be present in an R or S configuration, said R and S notation is used in correspondence with the rules described in Pure Applied Chem. 1976, 45, 11-13.

Reference to a compound or compounds described herein is intended to encompass that compound in each of its possible stereoisomeric forms and mixtures thereof unless the particular isomeric form is referred to specifically.

Another embodiment is a kit comprising a composition comprising a compound disclosed herein (e.g. a compound of Formula I), a container, and instructions for administration (e.g. prescribing and/or dosing instructions) of said composition to a subject for treatment of the dermatological condition or ocular condition and/or treatment (e.g. reduction) of inflammation associated with dermatological and/or ocular diseases, disorders, conditions, as described herein.

### EXAMPLES

The following examples are intended only to illustrate the methods, compositions, and compounds of the present disclosure and should in no way be construed as limiting the methods, compositions, and compounds of the present disclosure.

### Example 1

### Comparison of Potency of PDE4 Inhibition of Clinical PDE4 Compounds and the Compound of Formula I

A phosphodiesterase assay was performed using recombinant human PDE expressed in a baculoviral system. Enzyme has been tested for its similarity to PDE enzyme taken from human tissue using known inhibitor standards where available. The assay method was a modification of the method of Thompson and Appleman (Biochemistry 10; 311-316; 1971), which was been adapted for 96 well plate format.

The assay consisted of a two-step procedure. Tritium-labeled cyclic AMP was hydrolyzed to 5′-AMP by phosphodiesterase. The 5′-AMP was then further hydrolyzed to adenosine by nucleotidase in snake venom. An anion-exchange resin bound all charged nucleotides and left [³H]adenosine as the only labeled compound to be counted by liquid scintillation.

Four compounds—roflumilast, apremilast, AN-2728, GSK-256066, and Compound A—were tested against human PDE4B2. All compounds were tested at a 10 μM starting concentration with a 1 in 3, 8 point IC₅₀. The four compounds were then retested at 100 nM starting concentration with a 1 in 5, 8 point IC50. The IC₅₀ of each compound and the standard inhibitor (Rolipram) was determined. The standard inhibitor value was compared to historical assay data to ensure that it was within an acceptable range.

The compound of Formula I shows surprisingly more potency at PDE4 compared to approved PDE4 inhibitors, and the potency of the compound of Formula I is comparable to GSK PDE4 inhibitor GSK 256066. In addition, CHF-6001 exhibits greater than 50 fold higher selectivity for PDE4 versus other PDEs (see Table 1 below) indicating that this compound could potentially have lower tolerability and safety issues.

TABLE 1

| | | Enzymatic | |
|---|---|---|---|
| | | IC50 | PMBC TNF Inhib. |
| Entry | Compound | (nM) | IC50 (nM)* |
| 1 | 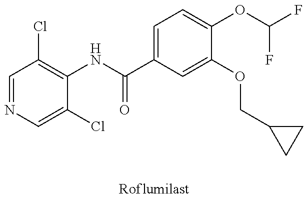 [SMILES: O=C(Nc1c(Cl)cncc1Cl)c1ccc(OC(F)F)c(OCC2CC2)c1] | 0.2-0.5 | 1.8 |
| 2 | 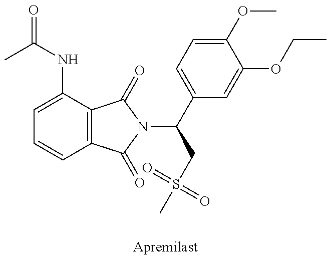 [SMILES: CCOc1cc([C@@H](CS(C)(=O)=O)N2C(=O)c3cccc(NC(C)=O)c3C2=O)ccc1OC] | 74 | 110 |
| 3 | 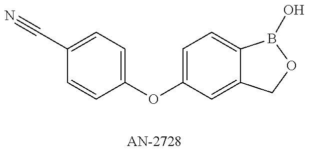 [SMILES: N#Cc1ccc(Oc2ccc3c(c2)COB3O)cc1] | 26 | 170 |
| 4 | 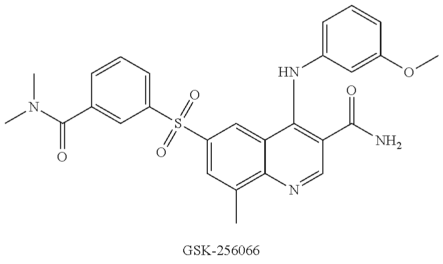 | 0.007 | 0.01 |
| 5 | 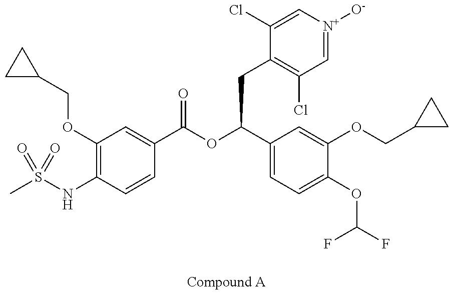 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1] | 0.04 | 0.03 |

Notes:
*Data from literature.

The compound of Formula I thus represents a potent and safer PDE4 inhibitor for the development of new therapeutic agents in dermatological and ophthalmic diseases with excessive inflammatory responses. Additional data supporting the superior efficacy and safety of the compound of Formula I in both dermatological and ocular inflammation animal models can be seen in the following additional examples.

### Example 2

### Effect of Topical Administration of the Compound of Formula I on Dermatitis Animal Model

Female SKH1-E hairless mice (Charles River) weighing approximately 25 g at 2-3 months of age were used in the following study. For model induction, the mice were sensitized on Day −7 (minus seven) by topical application of 50 μl of 5% Oxazolone (OXA) solubilized in ethanol on the hind flank. Dermatitis was elicited by repeated topical challenges with 50 μl of 0.1% Oxazolone every other day until the end of the study.

For administration of the test compounds, the test compounds were solubilized in a 70% ethanol/30% DMSO vehicle solution and kept at 4° C. Beginning on Day 0, the test compounds were administered twice daily (AM/PM) by topical application (50 μl volume) for 12 days. Betamethasone (0.12%) was used as a positive control and dosed once daily in the AM. Test article dosing and topical challenges were separated by at least 4 hours and all compounds were masked to investigators.

For clinical assessment, the degree of dermatitis was graded on a SCORAD (scoring atopic dermatitis) based scoring system of 0-3 (with 0=no symptoms, 1=Mild, 2=Moderate, 3=Severe) prior to challenge by a masked investigator.

For tissue collection, on the day of necropsy, animals were euthanized at 2 hours post final dose via CO₂ inhalation with death verified by cervical dislocation. Whole blood was collected via cardiac puncture (˜250 μl) in EDTA/K⁺ treated tubes for plasma analysis of drug concentrations. A rectangular strip of skin from the left side of treatment back area was collected for hematoxylin-eosin (H&E) stain. Prior to placing tissue in 10% formalin, the skin thickness was measured using a calibrated caliper (mm). Three 8 mm tissue punches were then collected from the right side of the treatment area and stored at −80° C. for cytokine protein (LUMINEX) or gene expression assays.

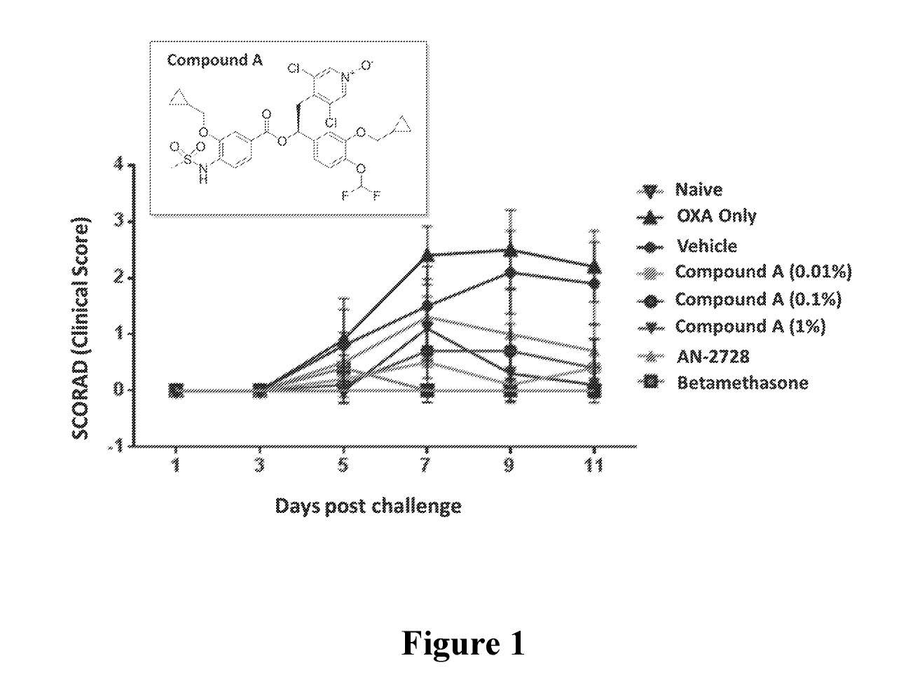

It was found that topically administered compound of Formula I (0.01-1% w/v) reduced SCORAD clinical scores 4 days earlier and significantly better than the benchmark PDE4 inhibitor compound AN-2728 in a dose dependent manner at a dose lower than AN-2728, as is shown in FIG. 1. The associated statistical significances of the compound of Formula I and AN-2728 as compared to the vehicle can be seen below in table 2.

TABLE 2
Statistics

| Entry | Drug vs. Vehicle | Day 5 | Day 7 | Day 9 | Day 11 |
|---|---|---|---|---|---|
| 1 | Compound A | p < 0.01 | p < 0.001 | p < 0.001 | p < 0.001 |
| | (0.01%) | | | | |
| 2 | Compound A | p < 0.01 | p < 0.01 | p < 0.001 | p < 0.001 |
| | (0.1%) | | | | |
| 3 | Compound A | p < 0.001 | n.s. | p < 0.001 | p < 0.001 |
| | (1%) | | | | |
| 4 | AN-2728 | n.s. | n.s. | p < 0.01 | p < 0.001 |

Abbreviations:
n.s.: no statistical significance

As can be seen from FIG. 1 and Table 2, the compound of Formula I showed a statistically significant improvement in SCORAD clinical scores by day 5, whereas AN-2728 did not show a statistically significant improvement in SCORAD clinical scores until at least day 9.

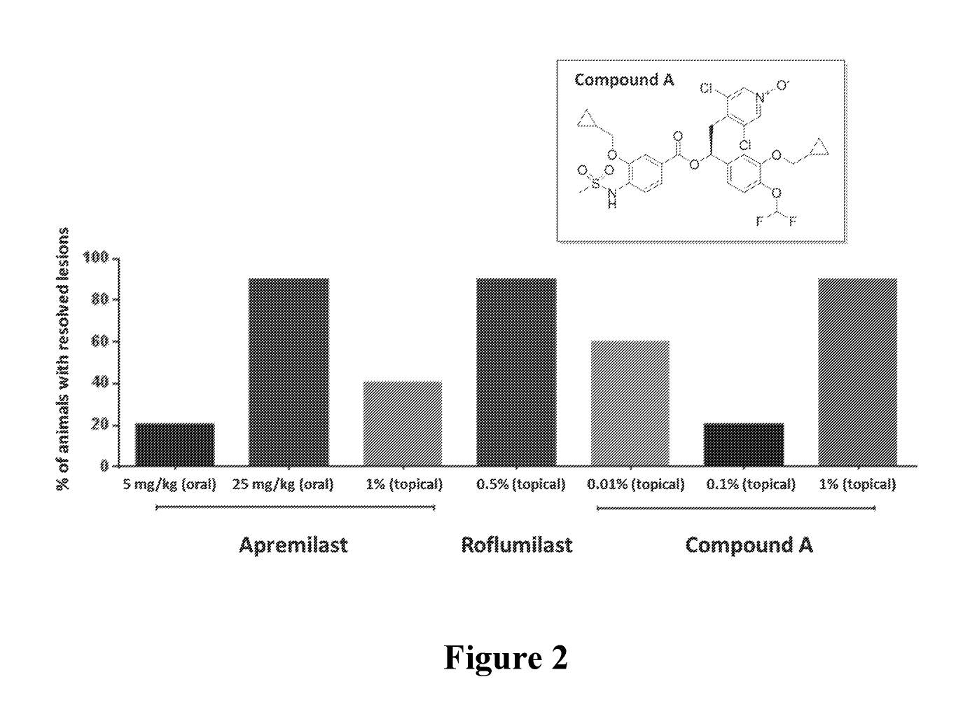

In addition, it was also found that the topical efficacy of the compound of Formula I was surprisingly far superior when compared to oral PDE4 inhibitors such as Apremilast and Roflumilast, as is shown in FIG. 2. In particular, it can be seen that the compound of Formula I topically applied at 1% demonstrated comparable efficacy (approximately 90% animals with resolved lesions) to orally administered apremilast when the apremilast was administered at a dose of 25 mg/kg, which is similar to the approved dose of 30 mg/kg for apremilast in the treatment of psoriasis in humans, and also demonstrated good efficacy (approximately 60% of animals with resolved lesions) was maintained even when the dose of the compound of Formula I was lowered by two orders of magnitude to 0.01%. Furthermore, topical administration of apremilast at 1% was shown to be surprisingly less effective than topical administration of the compound of Formula I at 1%, with the topical apremilast only showing approximately 40% of animals with resolved skin lesions whereas the compound of Formula I showed over twice the efficacy with approximately 90% of animals showing resolved lesions. Moreover, topical administration of the compound of Formula I at the low dose of 0.01% maintained good efficacy (approximately 60% of animals with resolved lesions) even when compared to the 50-fold higher dose of roflumilast at 0.5%.

### Example 3

### Demonstration of Lower Systemic Exposure of the Compound of Formula I

The whole-blood samples collected in Example 2 were analyzed for drug concentrations. In particular, plasma samples were extracted by protein precipitation with acetonitrile and analyzed for drug concentrations by the bioanalytical laboratory using liquid chromatography tandem mass spectrometry (LC-MS/MS). Propranolol (200 ng/mL) was used as an internal standard for the extraction procedure. Working standards were prepared by series dilution (0.5 ng/mL) into acetonitrile from a stock solution of 10 ug/mL. All samples were analyzed using the API 5000 LC-MS/MS system in 0.1% formic acid in water.

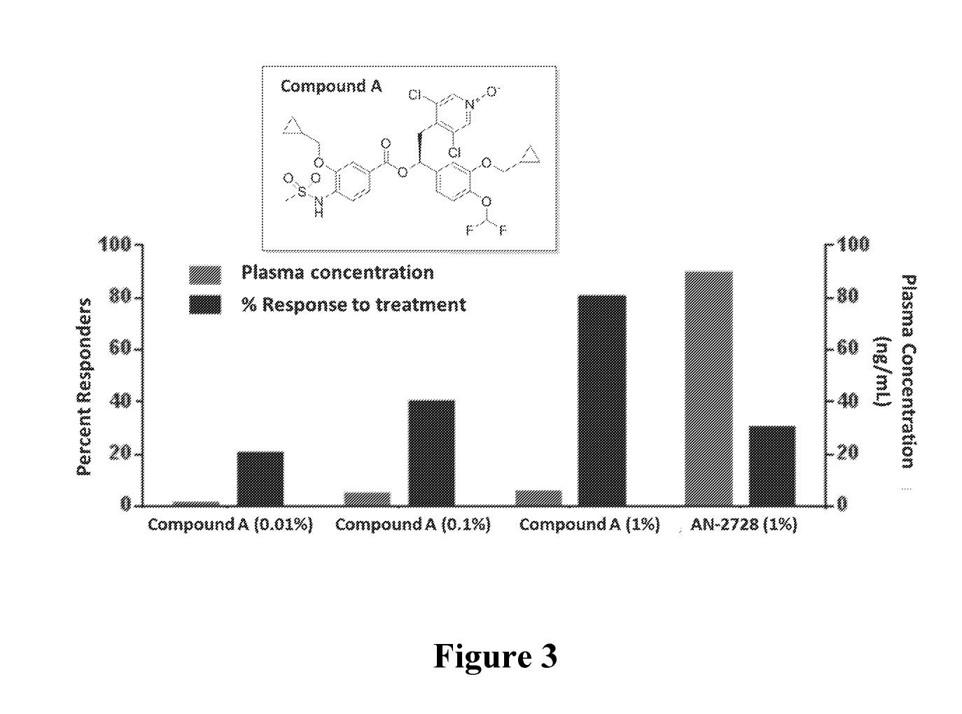

It was found that efficacy was achieved with low systemic exposure with plasma concentrations of less than 5 ng/mL when delivered topically to the skin, as can be seen in FIG. 3. In the figure, the “% Response to treatment” indicates the percentage of the group with minimal to moderate dermatitis score via hematoxylin-eosin (H&E) stain (see Example 2). For groups of animals tested with Compound A at 0.1% and 1%, and for the group of animals tested with AN-2728, there were 9 to 10 animals per group for which data was collected. For the groups of animals tested with Compound A at 0.01%, there were 2 animals in that group for which data was collected because 8 of the 10 animals in that group were below quantifiable limits.

Applicants have also observed that the compound of Formula I does not show any oral bio-availability, which is expected to contribute to lower systemic exposure and higher tolerability.

### Example 4

### Effect of the Compound of Formula I on Ocular Inflammation Model

Male Lewis rats, weighing about 250 to 270 grams, were purchased from Charles River and used for all studies. Lipopolysaccharide (LPS) was made fresh in sterile saline (1 mg/ml), kept on ice, and administered by footpad injection at a total dose of 100 μg (100 μl) per animal. Twenty-four hours later, at the time of maximal inflammation in this model, the rats were euthanized by CO₂ inhalation. Aqueous humor was collected and analyzed for protein concentration and cell counts.

For the calculation of the percentage of cell inhibition, the number of cells in treatment group were divided by the average number of cells in vehicle group times 100. For the calculation of the percentage of protein inhibition, the amount protein in treatment group was divided by the average protein in the vehicle group times 100.

Aqueous humor was collected from both eyes by anterior chamber puncture, performed under an operating microscope, using a 30-gauge needle and 25 μl Hamilton syringe (Catalog number 1702). aqueous humor from both eyes was pooled and kept on ice until further analysis.

Protein determination was carried out using a standard Bradford method. Cell counting was done by using either a hemocytometer or an automated cell counter (BioRad TC10).

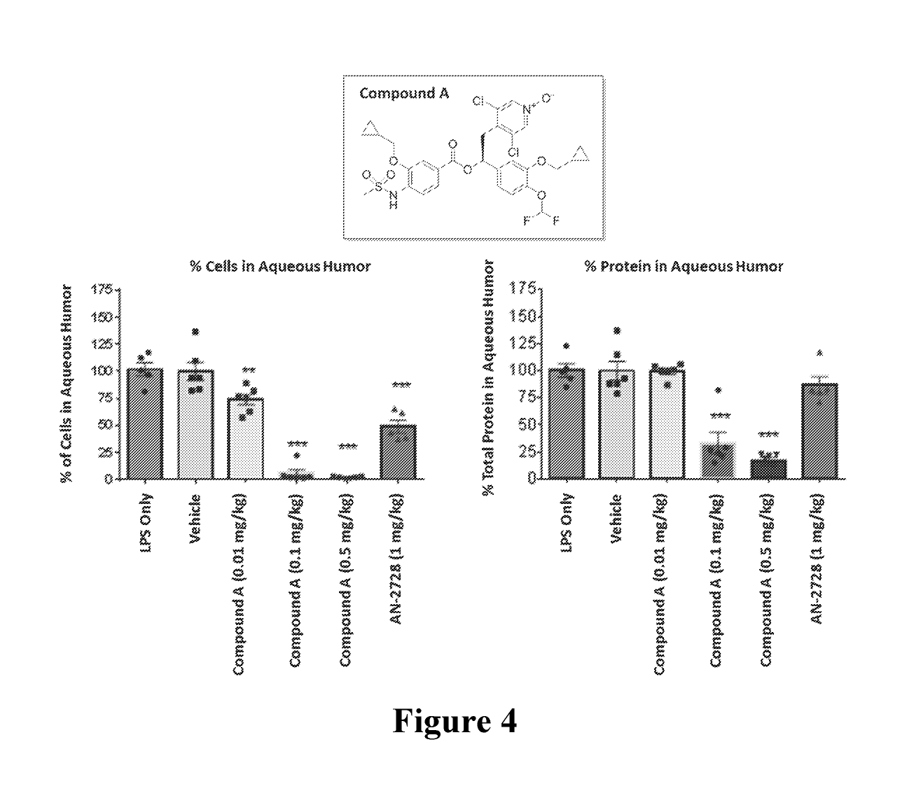

The compound of Formula I significantly reduced the percentage of cells and protein in the aqueous humor in a dose dependent manner when administered 0.01-0.5 mg/kg subcutaneously, as is shown in FIG. 4. In the figure, the squares, circles, and triangles show additional data points above the mean (scatter graph). The statistical significances are p<0.05 (*), p<0.01 (**), and p <0.001 (***). The efficacy of the compound of Formula I was superior to that of AN-2728.

Surprisingly, the compound of Formula I showed superior efficacy and safety in the ocular inflammation models when compared to other PDE4 inhibitors. Notably, the PDE4 inhibitor GSK 256066 had comparable potency, but resulted in death of the animals (Table 3).

TABLE 3

| | | | | % | |
|---|---|---|---|---|---|
| | | Systemic | | Efficacy | |
| | | Dose | % | Cell % | Protein % |
| Entry | Compound | (mg/kg) | Survival | Inhibition | Inhibition |
| 1 | 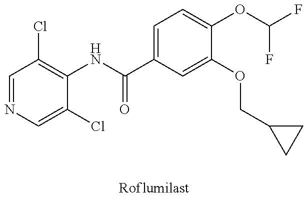 [SMILES: O=C(Nc1c(Cl)cncc1Cl)c1ccc(OC(F)F)c(OCC2CC2)c1] | 0.3 | 100% | 97% | 77% |
| 2 | 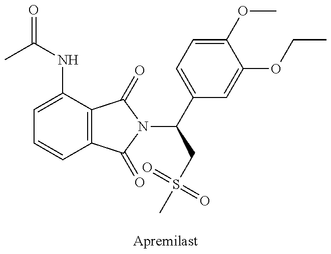 [SMILES: CCOc1cc([C@@H](CS(C)(=O)=O)N2C(=O)c3cccc(NC(C)=O)c3C2=O)ccc1OC] | 10.0 | 100% | 94% | 58% |
| 3 | 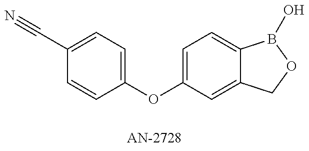 [SMILES: N#Cc1ccc(Oc2ccc3c(c2)COB3O)cc1] | 1.0 | 100% | 51% | 13% |
| 4 | 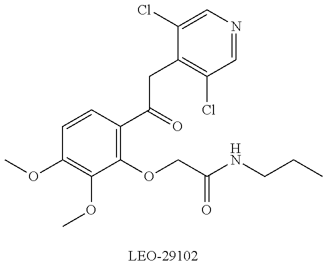 [SMILES: CCCNC(=O)COc1c(C(=O)Cc2c(Cl)cncc2Cl)ccc(OC)c1OC] | 1.0 | 100% | 67% | 29% |
| 5 | 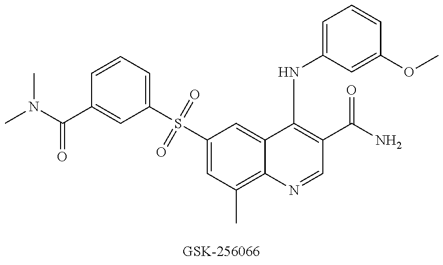 | 1.0 | 0% | N/A | N/A |
| 6 | 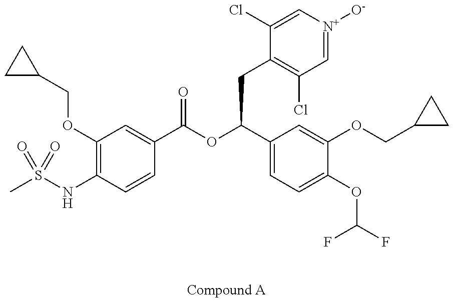 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1] | 0.1 | 100% | 95% | 67% |

Abbreviations:
N/A: not applicable

### Example 5

### Example of Treatment of Plaque Psoriasis

A 58-year old male presents with erythematous plaques covered with thick, silvery, shiny scales on the extensor surfaces of his elbows and knees. After a clinical evaluation, the patient is diagnosed with plaque psoriasis. The patient is prescribed a pharmaceutical composition containing an effective amount of the compound of Formula I with instructions to apply the composition topically to the plaques up to three times a day. After 1 to 4 months of applying the composition the patient reports the plaques have almost completely disappeared.

### Example 6

### Example of Treatment of Dry Eye Disease

A 71-year old female presents with a history of having gritty, itchy and burning sensations in the both eyes which was previously generally diagnose as dry eye and has been treated with artificial tear drops applied topically to the eye as needed. The patient complains that the artificial tear solutions require too many applications per day. Medical evaluation of the patient results in the identification of the patient's dry eye disease as being dry eye disease where tear production is presumed to be suppressed due to ocular inflammation associated with the dry eye disease. The patient is prescribed eye drops containing an effective amount of the compound of Formula I with instructions to apply medication once a day. After about 1 to 2 months of applying the medication, the patient reports enough of an increase in tear production that the artificial tear drops are needed much less frequently and also a significant reduction in the gritty, itchy and burning sensations that accompanied the dry eye disease.

### Example 7

### Example of Treatment of Rosacea

A 43-year old male presents with visible facial erythema and edema with telangiectases and visible papules and pustules resembling acne. The patient indicates that these symptoms have developed before, often in association with stress at work and often triggered by eating spicy food, as is the situation with the present signs. Upon clinical examination, the patient is diagnosed with rosacea in its inflammatory phase. The patient is prescribed a topical composition comprising an effective amount of the compound of Formula I and instructed to apply the composition to the affected areas one or twice a day. After about two days of applying the composition as instructed, the patient reports noticeable clearing of the affected areas, and after a few additional days of applying the composition, the affected areas are much cleared and the pustules and papules are no longer present.

### Example 8

### Example of Treatment of Pruritus

An 8-year old female presents with a history of chronic relapsing atopic dermatitis (eczema), with severe itching, beginning at 8 months of age. Her rash is characterized by red, itchy eruptions on cheeks and legs. Patient's parents stated that during a flare-up child is irritable at home and severe scratching disrupts sleep at night. Patient also feels very self-conscious of rash and is socially withdrawn at school during a flare-up. History of treatment have included lukewarm soaking baths and the use of over the counter emollients and creams. Topical calcineurin inhibitors were reported to exacerbate pruritus. The patient is prescribed a topical cream of the compound of Formula I with instructions to apply medication once day. Within the first week of using the cream, patient and patient's parents report a noticeable reduction in scratching. After approximately 3 weeks, a significant reduction in redness and eruptions on the skin of the face and legs were also observed.

Throughout this specification reference is made to publications such as US and foreign patent applications, journal articles, book chapters, and others. All such publications are expressly incorporated by reference in their entirety, including supplemental/supporting information sections published with the corresponding references, for all purposes unless otherwise indicated.

The foregoing descriptions details methods, compositions, and compounds that can be employed to treat dermatological disorders and ocular disorders, and represents the best mode contemplated. It should not be construed as limiting the overall scope hereof; rather, the ambit of the present disclosure is to be governed only by the lawful construction of the appended claims.

What is claimed:

1. A method of treating a dermatological condition or ocular condition in an individual in need thereof, the method comprising administering an effective amount of a compound of Formula Ia: 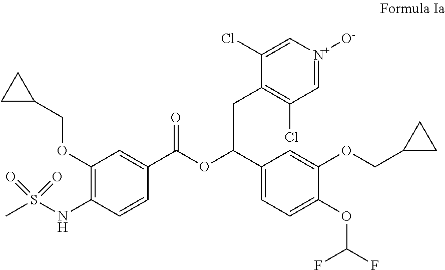 [SMILES: CS(=O)(=O)Nc1ccc(C(=O)OC(Cc2c(Cl)c[n+]([O-])cc2Cl)c2ccc(OC(F)F)c(OCC3CC3)c2)cc1OCC1CC1]
to the individual in need thereof.

2. The method of claim 1, wherein the dermatological condition is selected from the group consisting of atopic dermatitis, acne, psoriasis, seborrheic dermatitis, actinic keratosis, actinic keratosis, melisma, post-inflammatory hyperpigmentation, pruritus, and rosacea.

3. The method of claim 2, wherein the dermatological condition is psoriasis.

4. The method of claim 1, wherein the ocular condition is selected from the group consisting of uveitis, dry eye disease, keratitis, allergic eye disease, infectious keratitis, herpetic keratitis, retinitis, choroiditis, Behcet's disease, and wet and dry age-related macular degeneration (ARMD).

5. The method of claim 4, wherein the ocular condition is dry eye disease.

6. The method of claim 5, wherein the dry eye disease is dry eye disease in which tear production is suppressed due to ocular inflammation.

7. The method of claim 1, wherein the administering of the compound of Formula Ia reduces inflammation associated with the dermatological condition or the ocular condition.

8. The method of claim 1, wherein the compound of Formula Ia is administered topically.

9. The method of claim 1, wherein the compound of Formula Ia has the structure:

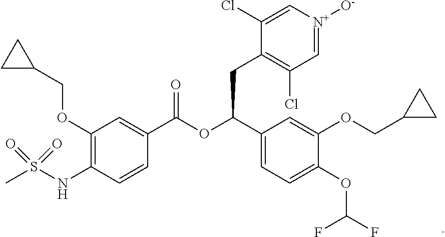 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

10.-40. (canceled)
